(19) United States
(12) Patent Application Publication
(10) Pub. No.: US 2022/0249031 A1
(43) Pub. Date: Aug. 11, 2022

(54) DEEP END-TO-END CLASSIFICATION OF ELECTROCARDIOGRAM DATA

(71) Applicant: Oxford University Innovation Limited, Oxford (GB)

(72) Inventors: David CLIFTON, Oxford (Oxfordshire) (GB); Tingting ZHU, Oxford (Oxfordshire) (GB); Girmaw John Abebe TADESSEE, Oxford (Oxfordshire) (GB)

(21) Appl. No.: 17/629,446

(22) PCT Filed: Jul. 22, 2020
(86) PCT No.: PCT/GB2020/051747

§ 371 Date: Jan. 24, 2022

(30) Foreign Application Priority Data
Jul. 25, 2019 (GB) 1910657.4

(51) Int. Cl. A61B 5/00; A61B 5/35

(52) U.S. Cl. CPC A61B 5/7267; A61B 5/35; A61B 5/7246; A61B 5/7257

(57) ABSTRACT

There is disclosed a computer-implemented method of classifying electrocardiogram data of a patient, comprising the steps of receiving input data from each of a plurality of electrocardiogram leads, arranging the input data into a single combined image, and applying a machine-learning algorithm to the combined image to classify the electrocardiogram data.

The invention relates to computer-implemented methods of using machine-learning algorithms to categorise electrocardiogram data.

One of the most challenging issues facing global societies is the delivery of healthcare to an ageing and expanding population. Chronic diseases are the leading cause of death for both developed and developing countries, representing 70% of all deaths, and cardiovascular disease (CVD) accounts for most of these (17.9 million annually) [1]. It is estimated that 85% of CVD deaths are due to heart attacks (i.e., myocardial infarctions) and strokes. Traditional diagnosis of CVDs such as myocardial infarction (MI) mainly employs interpretation of electrocardiogram (ECG) recordings and blood tests, which requires precise acquisition devices and clinical expertise. Diagnosis is difficult to achieve in a timely manner due to the slow generation of results from laboratory tests, as well as the inter-observer variability in ECG interpretation resulting in disagreement of diagnosis.

Particularly in the case of an ambulatory setting, where only ECGs are available, pre-diagnosis of MI would better prepare clinicians to make treatment decisions. In order to address these challenges, research on automated algorithms using ECG for heart disease classification serving as data-driven decision making tools is increasingly popular, with growing amounts of available ECG data being collected in wearable devices. However, most automated ECG analysis has relied on feature engineering, where hand-crafted features extracted from ECG waveforms are used for the purpose of heart disease classification. These features do not generalise well, potentially due to variation in acquisition settings such as sampling rate and mounting positions. These methods also require domain-specific knowledge, a large amount of effort to pre-process ECG data, and beat-extraction, which produces variant results depending on the algorithm used for analysis [2]. For the detection of ST-elevated MI, previous hospital-wise clinical studies have demonstrated the feasibility of utilising automated algorithms, which achieve a sensitivity of 65% and specificity of 90% or an accuracy around 70% [3], [4].

Deep neural networks (DNNs) have become increasingly popular in the domain of ECG analysis. Existing deep neural networks can extract features from ECG automatically without domain-specific knowledge. There are certain cases where DNNs outperform clinical experts [5], [6], [7]. Most experiments in the literature rely on the use of publicly-available datasets, placing a constraint on the range of applications which can be proposed. This results in domain-specific DNNs purposely designed for the detection of, for example, arrhythmia [5], [8], atrial fibrillation [9], heartbeat classification [10], or serving as a general purpose abnormal ECG detector [7]. While most models of this type focus on arrhythmia detection using single lead ECG (e.g., [8]), clinical practice for ECG evaluation of heart disease such as MI requires the inspection of 12-lead ECGs. Thus, any comparison between performance of clinicians and DNNs is unfair as clinical expertise is not trained or developed on single lead ECG analysis.

In view of these limitations, there is still a need for providing methods using machine learning algorithms with improved ability to classify ECG data, particularly for detection of heart disease. Therefore, it is an object of the invention to provide an improved method for classifying ECG data that is more accurate and robust.

The model disclosed herein is validated on a large cohort of over 15,000 patients. The best-performing embodiment demonstrates that it is robust in performing heart disease classification, with an improvement of 9.0% in accuracy when compared to the next best-performing alternative embodiment investigated.

According to an aspect of the invention, there is provided a computer-implemented method of classifying electrocardiogram data of a patient, comprising the steps of receiving input data from each of a plurality of electrocardiogram leads, arranging the input data into a single combined image, and applying a machine-learning algorithm to the combined image to classify the electrocardiogram data.

Applying the machine-learning algorithm to the combined data from multiple ECG leads means that correlations between the data from different leads can be taken advantage of to improve classification of the ECG data of the patient. Arranging the input data in an image format allows for the use of algorithms optimised for analysis of image data, and for transfer learning from neural networks trained on large image datasets.

In an embodiment, the plurality of electrocardiogram leads comprises twelve leads, the twelve leads comprising three limb leads, three augmented limb leads, and six precordial leads. Using a full standard 12-lead ECG arrangement means that the maximum amount of data can be used by the algorithm, further improving the accuracy and robustness of the classification. It also ensures compatibility with standard ECG measurements taken in a clinical setting.

In an embodiment, the input data are arranged in the combined image either in a grid of four columns and three rows, wherein the first column contains the input data from the three limb leads, the second column contains the input data from the three augmented limb leads, and the third and fourth columns each contain the input data from three of the six precordial leads, or in a grid of four rows and three columns, wherein the first row contains the input data from the three limb leads, the second row contains the input data from the three augmented limb leads, and the third and fourth rows each contain the input data from three of the six precordial leads. Arranging the input data in this manner has been shown to provide the most accurate output classification, even over a variety of different implementations of the machine-learning algorithm.

In an embodiment, the machine-learning algorithm comprises a deep neural network. Deep neural networks are well-established tools for image analysis, and so are well-suited to classifying data in the format used by embodiments of the disclosure.

In an embodiment, the deep neural network comprises one or more autoencoder layers configured to perform feature extraction on the combined image to produce a representation of the combined image with lower dimensionality than the combined image. Using an autoencoder to perform feature extraction reduces the dimensionality of the input data and extracts the most significant features characterising the ECG data. This allows the classification of the input data in a more efficient manner that is less prone to overfitting when trained on a particular dataset.

In an embodiment, the deep neural network is trained by minimising a reconstruction error of the autoencoder layers. Minimising a reconstruction error of the autoencoder ensures that the representation with reduced dimensionality most accurately reflects the characterising features of the ECG data.

In an embodiment, the neural network is a convolutional neural network, and the one or more autoencoder layers comprise one or more convolutional layers. Convolutional neural networks can take account of spatial local structure in an input image, and so allow for more accurate encoding of the input data prior to classification.

In an embodiment, the deep neural network further comprises one or more classification layers, configured to classify the electrocardiogram data using the representation of the combined image, and the deep neural network is trained by minimising a joint error calculated by combining a reconstruction error of the autoencoder layers and a classification error of the classification layers.

Minimising a joint error of the autoencoder and classification layers means that the machine-learning algorithm is optimised for the overall process of analysing and classifying the input data. This improves the accuracy of the result relative to separately optimising the classification and autoencoder layers, where the features extracted by the autoencoder layers to represent the data may not be those most relevant for classifying the data.

In an embodiment, combining the reconstruction error and the classification error comprises combining the classification error with a normalised reconstruction error. Although classification error is typically expressed in a logarithmic scale, reconstruction error as expressed by many typical methods can take a range of values larger than one. Normalising the reconstruction error ensures that the relative significance of the reconstruction and classifications errors is properly accounted for in the joint error.

In an embodiment, the machine-learning algorithm is trained using electrocardiogram data of a plurality of patients. Using electrocardiogram data to train the algorithm has been shown to produce more accurate results than when the algorithm is trained on other types of image data. This is particularly relevant when using autoencoder layers that may be trained on large image datasets.

In an embodiment, the machine-learning algorithm is configured to classify the electrocardiogram data into one of two or more categories, the two or more categories comprising normal heart activity and one or more categories of disease. The method may be used to diagnose a particular form of disease, such as heart disease. Depending on the training data used, it may also be used to indicate general pathological heart activity.

In an embodiment, the one or more categories of disease comprise myocardial infarction. Myocardial infarction is particularly suited to diagnosis using simultaneous analysis of data from a plurality of ECG leads.

In an embodiment, the step of arranging the input data into a single combined image comprises, processing the input data to produce a spectrogram of the spectrum of frequencies of the ECG signal derived from each of the plurality of electrocardiogram leads, and arranging the spectrograms into a single combined image. Analysis using spectrogram data has been shown to be more robust to variations in how the data is collected from a patient, such as changes in electrode position or sampling rate.

In an embodiment, the step of arranging the input data into a single combined image further comprises normalising the input data. Normalising the spectrogram data ensures that it has the same range of values as pixel values in an image, which simplifies handling of the data by machine-learning algorithms designed for image processing.

Embodiments of the invention will now be described, by way of example only, with reference to the accompanying drawings in which corresponding reference symbols represent corresponding parts, and in which:

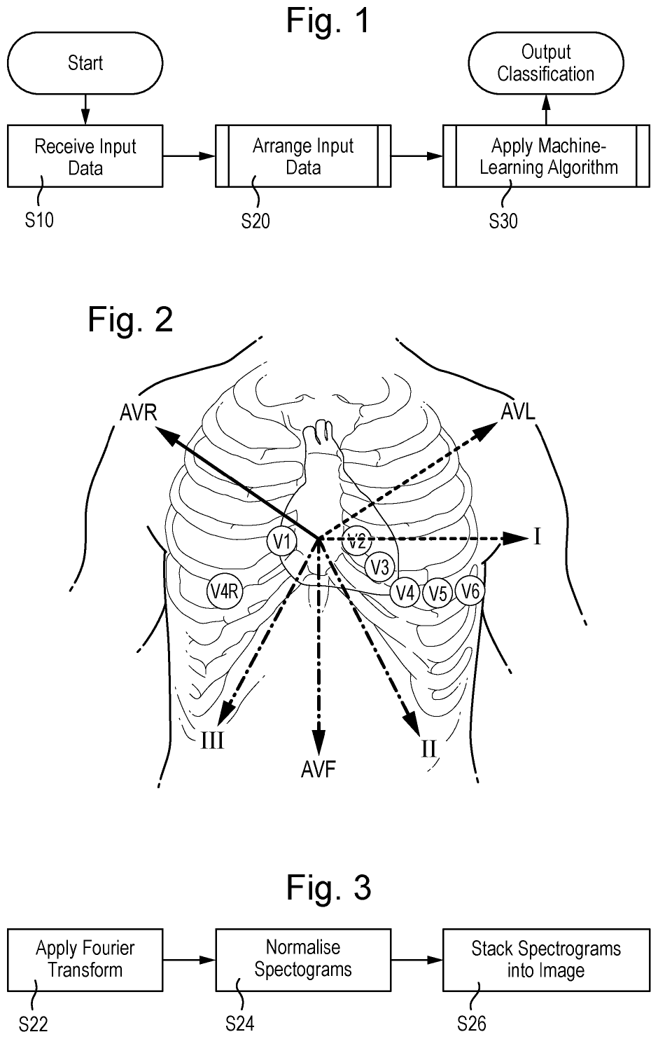

FIG. 1 is a flowchart illustrating the method disclosed herein;

FIG. 2 shows the placement of ECG electrodes in a standard 12-lead measurement;

FIG. 3 is a flowchart showing further detail of the step of arranging input data in an embodiment;

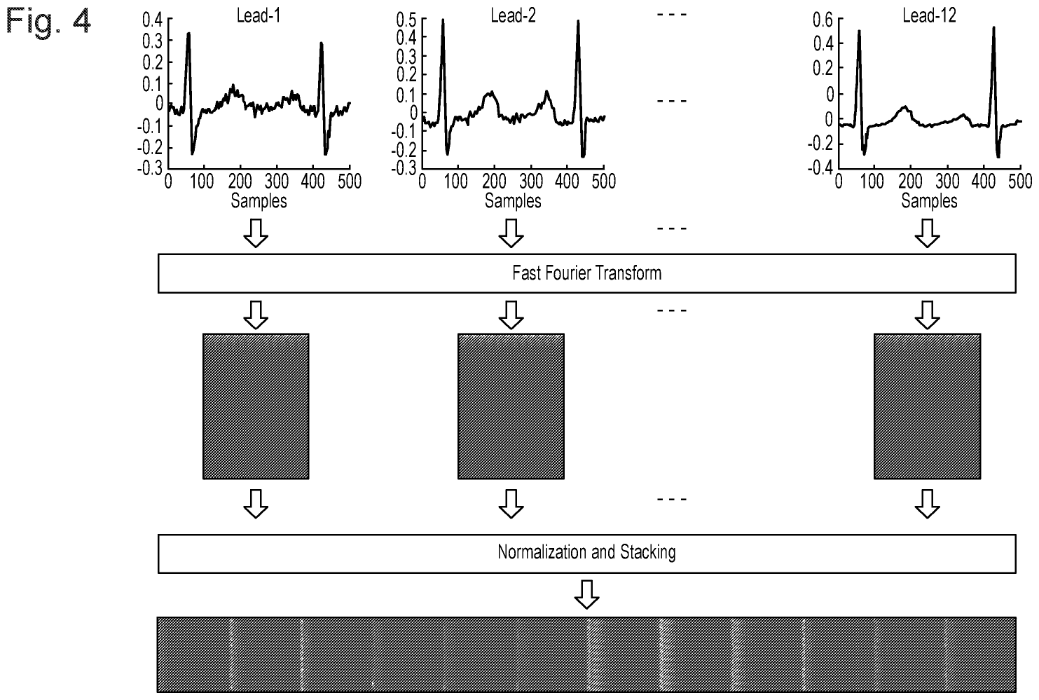

FIG. 4 shows how input data are processed for combining into the combined image in an embodiment;

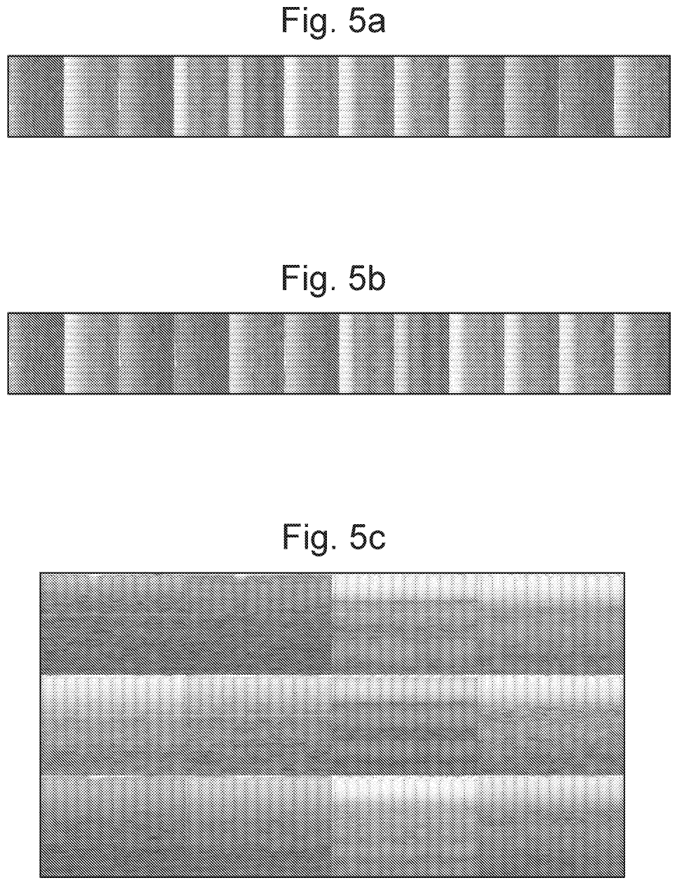

FIG. 5 shows three alternative arrangements of input data into a combined image;

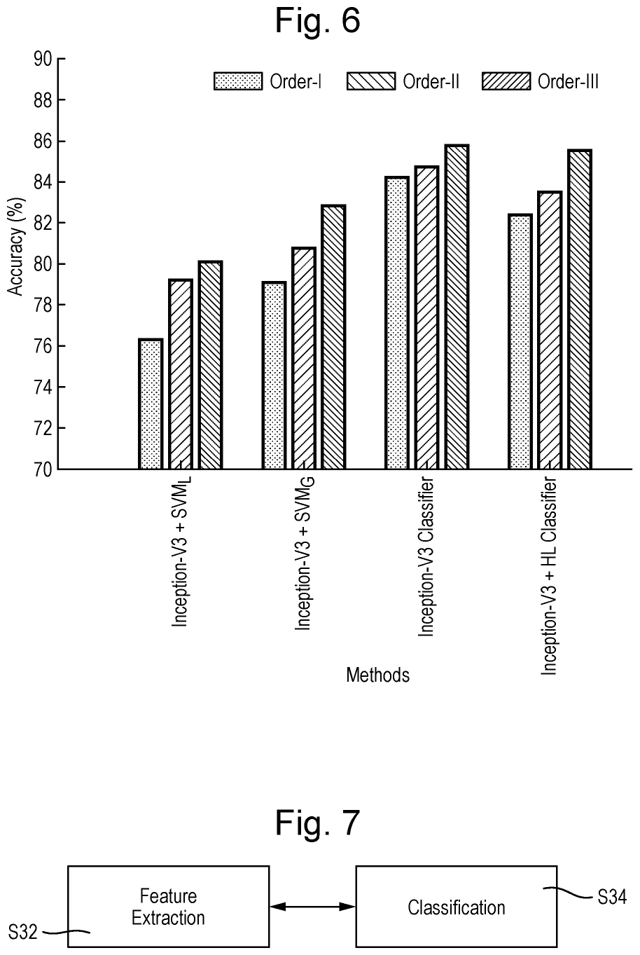

FIG. 6 shows a comparison of the accuracy of classification for the different arrangements of input data shown in FIG. 5 for several different choices of machine learning algorithm;

FIG. 7 is a flowchart showing further detail of the step of applying a machine-learning algorithm to the combined image in an embodiment;

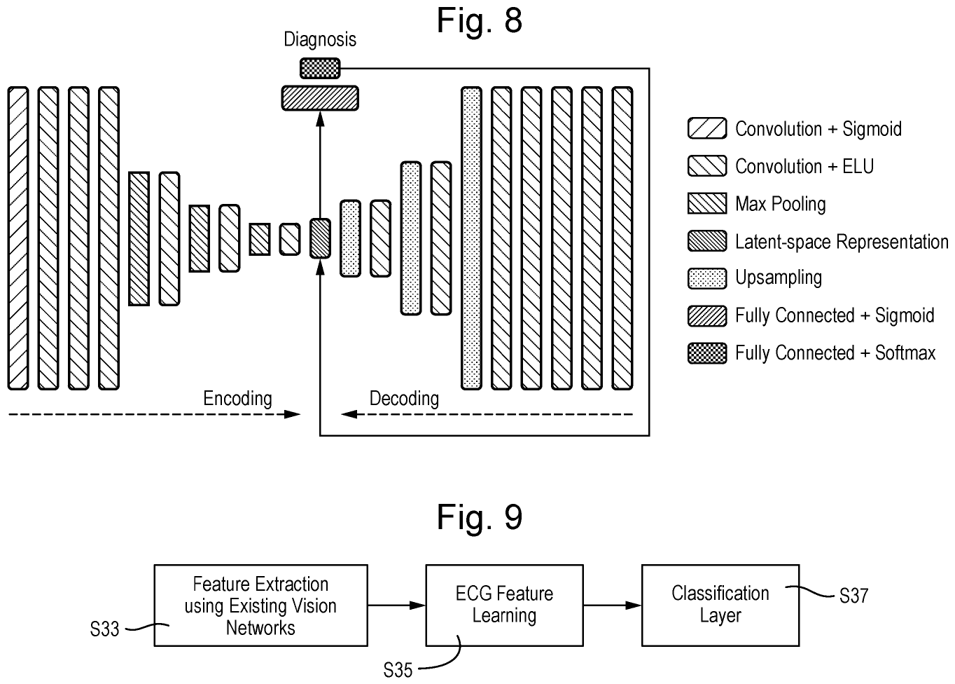

FIG. 8 shows detail of the neural network layers used in an embodiment;

FIG. 9 is a flowchart showing further detail of the step of applying a machine-learning algorithm to the combined image in an alternative embodiment to that shown in FIG. 7;

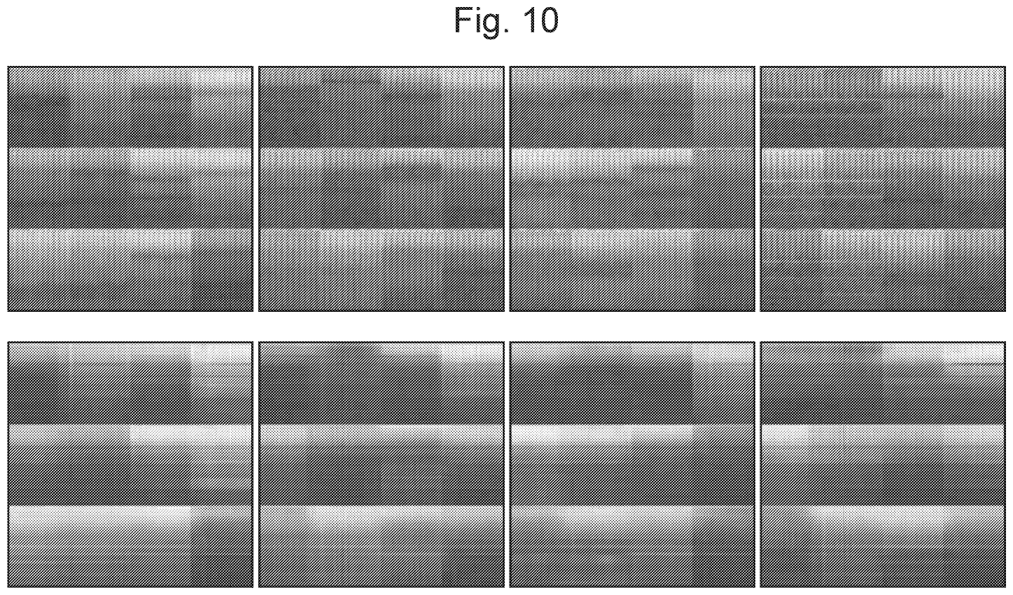

FIG. 10 shows examples of original combined image input data and reconstructed combined images from the decoder layers of an embodiment;

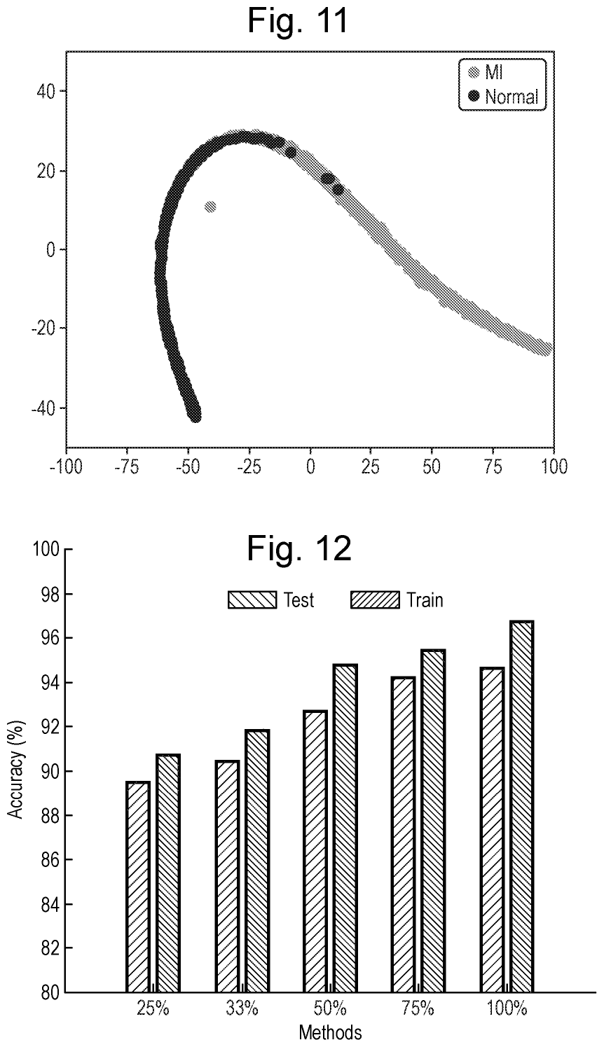

FIG. 11 shows a visualisation of the myocardial infarction vs. normal classes in the dense output of an embodiment;

FIG. 12 shows a comparison of the accuracy of results from an embodiment for different sizes of training data set.

Heart disease classification requires large amounts of patient data to train as well as parameter fine turning to achieve acceptable results in a clinical setting. In the case of MI, the PTB database [11] is commonly used for 12-lead ECG analysis [2]. Although most DNNs demonstrated high accuracy (≥80%) in the PTB dataset [12], [13], [14], [15], they were trained on thousands of ECG segments derived from a maximum of 150 patients. It is therefore not possible to evaluate the robustness of these DNNs when they are applied to a large cohort.

When dealing with an insufficient number of representative patients, one possible solution would be performing data augmentation [16], where synthetic data are generated via techniques such as data warping or generative adversarial networks. This approach is particularly suitable for images, speech, and activity recognition. However, it is challenging to apply data augmentation to 1D ECG traces for classification as the pathological information provided in each ECG waveform is limited and there is little time domain information available in a short-duration ECG recording (e.g., up to 10 seconds). Transfer learning has also been proposed as a means to address this limitation in small datasets. For ECG classification the weights of a previously-trained complex DNN based on a large dataset are retained and the classification layers are retrained for the new dataset [17], [18].

The previously discussed DNNs from the literature are variants of convolution neural networks (CNNs). Other types of DNNs, such as recurrent neural networks (RNNs) [19], have been used for modelling cardiac activity over a long period of time that are suitable for Holter monitoring. In cases where each ECG lead is shorter than ten seconds, there is little time domain information that is useful for RNNs. One approach to augment the time information is to treat ECG waveforms as images and perform automatic feature extraction via CNNs. When dealing with ECG input data as an image, DNNs like auto-encoders (AEs) can be used to extract high-level features. Denoising AEs are used for ECG signal enhancement [20], [21] and sparse AEs are considered for arrhythmia detection [22], [23], [24].

However, as discussed above, these approaches still have limitations in terms of their accuracy and robustness for large cohort sizes. To address these limitations, there is disclosed herein a method utilising a deep learning model for heart disease classification from simultaneous analysis of multiple lead ECGs. The method is a computer-implemented method of classifying electrocardiogram data of a patient, comprising the steps of receiving input data from each of a plurality of electrocardiogram leads, arranging the input data into a single combined image, and applying a machine-learning algorithm to the combined image to classify the electrocardiogram data

FIG. 1 shows a block diagram of the method. In step S10, input data are received from the plurality of ECG leads. In step S20, all the ECG lead data from an individual are combined to form a representative “image”. In step S30, the image is then fed into the machine learning algorithm for providing diagnosis of heart disease.

A standard 12-lead ECG is made up of the three standard bipolar limb leads (I, II and III), the three augmented limb leads (aVR, aVL and aVF), and the six precordial leads (V1, V2, V3, V4, V5 and V6). Their corresponding electrodes are mounted as shown in FIG. 2. Therefore, in an embodiment, the plurality of electrocardiogram leads from which input data are received comprises twelve leads, the twelve leads comprising three limb leads, three augmented limb leads, and six precordial leads.

The raw signal data from the plurality of ECG leads may be used directly by the machine-learning algorithm, in which case receiving input data from each of the plurality of leads comprises receiving the raw ECG signal data. In an embodiment, the raw signal data for each ECG lead comprises measurements of voltage on the electrode of the ECG lead as a function of time.

In an embodiment, arranging the input data into a single combined image comprises stacking the raw signal data. For example, in an embodiment where 12-lead ECG data are used, the input data may be arranged as an image of stacked raw 12-lead ECG signals.

### Fourier Transform and Normalisation

Arranging the input data into a single combined image may comprise performing further processing on the input data. In particular, in an embodiment, the step of arranging the input data into a single combined image comprises processing the input data to produce a spectrogram of the spectrum of frequencies of the ECG signal derived from each of the plurality of electrocardiogram leads, and arranging the spectrograms into a single combined image.

In the embodiment shown in FIG. 3, each ECG lead signal is converted into a spectrogram in step S22, before being stacked to form the image in step S26. Where spectrograms are used, each spectrogram is the spectrum of frequencies of the ECG signal derived from a single lead. Spectrogram representation has demonstrated its ability to improve robustness against variation in sampling rate and mounting positions of wearable sensors [25], [26]. This approach also helps to reduce the amount of data required for training.

In an embodiment, the spectrograms are calculated by applying a fast Fourier transform to the raw input data from the ECG leads. FIG. 4 shows an example of such processing applied to the raw input data. In the embodiment shown in FIG. 4, the time resolved raw signal from each lead is segmented into multiple windows, and its frequency-time (spectrogram) representation is obtained by applying a fast Fourier transform (FFT), [symbol] (·) to each window. The windows may be chosen so that the signal from the ECG lead is divided into a series of segments each containing one or more heart beats. The windows may all be chosen to have the same duration. In an embodiment where each window contains one heart beat signal, the windows may each be centred on the heart beat signal. If Eₙⁱ is the nth window of the ith ECG lead, the spectrograms after the FFT can be presented as Ēₙⁱ= [symbol] (Eₙⁱ).

The spectrogram contains the frequency response magnitude at different frequency bins for each window. Therefore, the spectrogram for each ECG lead comprises a 2D colour plot, with the frequency bins along one axis, the windows along the second axis, and the magnitude of the frequency component in each bin for each time window displayed using pixel colours.

In an embodiment the step of arranging the input data into a single combined image further comprises normalising the input data. This ensures that all of the input data from the different leads has the same maximum and minimum values, so that it is accounted for by the machine learning algorithm. The normalisation may consist of multiplying the signal from each ECG lead by a constant factor and/or adding a constant offset to the signal, such that the maximum and minimum values of the signals from each lead after normalisation are the same as those of the other leads. Normalisation may be applied to raw signals in embodiments which directly use raw signals, or to the spectrograms obtained by processing the raw signals.

In the embodiment of FIGS. 3 and 4, normalization of Ēₙⁱ in step S24 is performed as

$${\dot{E}}_{n}^{i} = \frac{{\overline{E}}_{n}^{i}}{max ( {E}_{n}^{i} )} \times 255.$$

Ėₙⁱ exhibits image-like characteristics as the normalization bounds its values to

[0,255]. This is the range of values that may be expected for typical image data used to train existing computer vision algorithms for feature extraction, and therefore normalising the data in this way makes inputting the data into such algorithms more straightforward.

Arranging the input data into a combined image by stacking the input data, and in particular the spectrograms, results in an image-like representation from ECG waveforms that enables transfer learning from existing vision networks that are pre-trained on large image datasets; e.g., ImageNet [27], and generates a high-dimension feature representation before classification.

Arranging the input data into a single combined image allows the machine learning algorithm to take account of the data from all of the available ECG leads simultaneously. This is advantageous compared to existing approaches that process the data from each lead separately. Processing all of the data together allows the algorithm to take account of correlations between the data from different leads, and leads to improved accuracy and robustness of classification.

### Stacking Order

Currently there is no standardisation for the display of 12-lead ECG waveforms in a clinical setting, and therefore their display order may vary depending on the manufacturer of particular ECG equipment. While it is expected that some ECG leads are highly correlated, the inventors have found that different polar orientations and ordering of ECG leads affect the accuracy of ECG classification.

In an embodiment, the input data are arranged in the combined image either in a grid of four columns and three rows, wherein the first column contains the input data from the three limb leads, the second column contains the input data from the three augmented limb leads, and the third and fourth columns each contain the input data from three of the six precordial leads, or in a grid of four rows and three columns, wherein the first row contains the input data from the three limb leads, the second row contains the input data from the three augmented limb leads, and the third and fourth rows each contain the input data from three of the six precordial leads.

The ECG leads are divided by cardiologists into four subgroups, each representing a vertical stacking of three leads: G₁=[I, II, III]^{T}, G₂=[V1, V2, V3]^{T}, G₃=[V4, V5, V6]^{T} and G₄=[aVL, aVR, aVF]^{T}, where T indicates ECG lead outputs are stacked as a column vector. In an embodiment, the third column (or third row) of the grid described above contains the precordial leads V1, V2, and V3, and the fourth column (or fourth row) contains the precordial leads V4, V5, and V6.

Three specific arrangements of stacked spectrograms (denoted as Order-I, Order-II and Order-III) were compared:
(i) Order-I=[G₁, G₂, G₃, G₄]^{T};
(ii) Order-II=[G₁, G₄, G₂, G₃]^{T}; and
(iii) Order-III=[G₁, G₄, G₂, G₃].

Order-III is a specific embodiment of the grid arrangement described above. Note that Order-III stacks the subgroups as a row vector as compared to Order-II. FIG. 5 shows visualisations of the three different stacked arrangements for displaying conventional 12-lead ECG spectrograms. Note that Order-I and Order-II are rotated by +90° in FIG. 5 for ease of display.

The three stacking arrangements, i.e., Order-I, Order-II, and Order-III, were experimented on the dataset across different classification methods. These methods include Inception-V3+SVM_{L}, Inception-V3+SVM_{G}, Inception-V3 Classifier and Inception-V3+HL Classifier. Further details of this dataset and classification methods used to test the stacking arrangements are given in the experimental and machine-learning algorithm sections below. The results of the test set in FIG. 6 show the effect of different stacking orders on the classification performance for the testing dataset.

Results of Order-III across 4 methods were consistently superior over other stacking methods. This suggests that using the grid arrangement of input data, and in particular the specific stacking method of Order-III, provides a better encoding of spatial relationship among the leads. Furthermore, the grid arrangement benefits from its square-like representation, and the specific ECG lead orientation arrangement of Order-III is identical to the paper version that is used in a clinical setting. For the remaining results described herein from testing of different choices of machine learning algorithm, stacking method of Order-III was used.

Therefore, use of the combined data from a plurality of leads in a single image results in more accurate and robust classification, and reduces the data needed for training. The arrangement of spectrograms into a grid, and in particular the specific arrangement of

Order-III, is shown to be particularly advantageous, and leads to improved accuracy using a variety of different machine-learning algorithms.

### Machine Learning Algorithm

The method further comprises steps of applying a machine-learning algorithm to the combined image to classify the data. In an embodiment, the machine-learning algorithm comprises a deep neural network. As shown in FIG. 7, in an embodiment there are two main parts to this algorithm, namely feature extraction in step S32 and classification in step S34.

In some embodiments, the algorithm is a two-stage algorithm trained on the two parts separately. The input data are processed by the feature extraction layers, and the output of the feature extraction layers is sent to the classification layers. This means that the reconstruction error of the feature extraction components of the algorithm, and the classification error of the classification components of the algorithm are optimised separately. This approach allows for transfer learning from large image datasets for the feature extraction layers.

Alternatively, the algorithm may be a one-stage algorithm, also referred to as an end-to-end model or end-to-end algorithm, where the reconstruction error and classification errors are simultaneously optimised. In these embodiments, the output of the classification layers is used in the feature extraction, for example, to influence the parameters of the feature extraction layers during training. Therefore, as well as the output of the feature extraction layers being fed to the classification layers, the output of the classification layers is also fed back into the feature extraction layers. This is illustrated by the two-headed nature of the arrow in FIG. 7.

Embodiments demonstrating both of these alternatives are discussed further below.

1) Feature Extraction: In an embodiment, the deep neural network comprises one or more autoencoder layers configured to perform feature extraction on the combined image to produce a representation of the combined image with lower dimensionality than the combined image. Feature extraction using machine learning algorithms is frequently achieved using autoencoders. A traditional autoencoder (AE) is an unsupervised network that serves as a dimensionality reduction tool [28]. A single-layer AE is composed of an encoder and a decoder which are multilayer neural networks, and a central layer that is shared among them, known as the hidden layer. The hidden layer is the compressed latent-space representation of the input data. The goal of an AE is to encode the input data into this latent-space representation, such that it is possible to decode the representation back into its original form of the input as accurately as possible. To achieve this, the deep neural network is trained by minimising a reconstruction error of the autoencoder layers.

Assuming the d dimensional input to be x∈ [symbol] ^{d}, and the latent representation to be z∈ [symbol] ^{d′}, where d≠d′, the encoder of an AE describes their relationship via a non-linear

mapping function, f(x), as

z=f(x)=σ(Wx+b) (1)

where σ(·) is an element-wise activation function, W is the weight matrix with dimension d×d′ and b is a bias vector. In the decoder, a similar mapping function, g(x′), can be constructed as

x′=g(z)=σ(W′z+b′) (2)

where x′ is the reconstruction of the input x, W′ is the weight matrix with dimension d×d′ and b′ is a bias vector. The parameters of the AE, denoted as θ={W, W′, b, b′}, can be estimated by minimising the reconstruction loss (or reconstruction error), [symbol] (x, x′), as

$$\begin{matrix}θ = {argmin}_{θ} ℒ ( x , {x}^{′} ) \\ = {argmin}_{θ} ℒ ( x , g ( f ( x ) )\end{matrix} \qquad ( 3 )$$

The reconstruction loss is a measure of how accurately the decoder layers of the autoencoder are able to reconstruct the original input data from the latent space representation in the hidden layer.

### Convolutional Autoencoder

In comparison to an AE, a simple convolutional neural network (CNN) consists of three basic building blocks: the convolutional layer, the pooling layer and the classification layer [31]. The convolutional layer computes feature maps from the input by convolving it with filters. The pooling layer, often a max-pooling layer, serves a sample-based discretisation process where it performs dimension reduction of an input representation to reduce overfitting. The classification layer contains the fully-connected layer which combines the flattened features that are learned by the convolutional layers and feed them to a softmax or sigmoid function to predict class labels.

In an embodiment, the neural network is a convolutional neural network, and the one or more autoencoder layers comprise one or more convolutional layers. A traditional AE ignores the spatial local structure in an input, and a standalone CNN requires manual design of convolutional filters. Therefore, it is advantageous to combine these two types of neural network into a convolutional AE (i.e., ConvAE) [32]. ConvAE acts as a combination which benefits from both networks. ConvAE is different from a traditional AE as its weights in the network are shared among all data points of the input, preserving spatial locality as well as having fewer number of parameters than an AE. This allows for a better latent representation that is sensitive to transitive relations of features. ConvAE is also better than a standard CNN as the former can learn the optimal filters that minimises the reconstruction error of the latent-space representation.

The hidden representation z of the lth convolution layer or feature map can be estimated as

z^{l}=σ(W^{l}*x+b^{l}) (5)

where * denotes the 2D convolution and the reconstruction x′ in the decoder can be estimated as [32]

x′=σ(Σ_{l∈D}W′^{l}* z^{l}+b^{l}) (6)

where D indicates the group of latent feature maps.

2) Classification: In an embodiment, the deep neural network comprises one or more classification layers, configured to classify the electrocardiogram data. As discussed earlier, the classification layer of the CNN can be used to predict labels. In an embodiment, the hidden layer is connected with a fully connected layer to allow for classification. A softmax layer is then added as an activation function to the output layer of the classifier to assign probability for each class label. The number of units in the output layer is defined as the number of classes (i.e., class c=1, . . . , C).

For any input latent representation z that comprises a set of vector {zⱼ}, where j=1, . . . , K sample size, the probability of zⱼ belonging to class c is defined as

$${p}_{jc} = p ( {z}_{j} = c \vert x ) = \frac{exp {z}_{j}}{{\sum }_{c = 1}^{C} exp {z}_{C}} \qquad ( 7 )$$

In an embodiment, the classification loss (or classification error) [symbol] _{ce}, is defined as the loss function of cross-entropy as

$${ℒ}_{ce} = - \frac{1}{K} {\sum }_{j = 1}^{K} {\sum }_{c = 1}^{C} {y}_{jc} log {p}_{jc} \qquad ( 8 )$$

where y_{jc} indicates the true cth class label that is assigned to the jth element of z. Other choices of classification loss are possible depending on the specific embodiment chosen. The classification loss is a measure of how accurately the machine-learning algorithm classifies the input data compared to the ‘true’ classifications, which may be determined from classifications by human operators. In an embodiment, the deep neural network is trained by minimising a classification error of the classification layers.

The classes may be chosen so that the classification layers are able to classify the input data to indicate whether the patient is suffering from, or at risk of, any disease that can be detected from ECG data. In particular, ECG data are often used to classify heart disease. In an embodiment, the machine-learning algorithm is configured to classify the electrocardiogram data into one of two or more categories, the two or more categories comprising normal heart activity and one or more categories of disease. In an embodiment, the one or more categories of disease comprise one or more categories of heart disease. The one or more categories of heart disease may include arrhythmia, atrial fibrillation, myocardial infarction.

The method described herein has been found to be particularly advantageous when used to classify ECG data as either normal or indicating myocardial infarction. Therefore, in an embodiment, the one or more categories of heart disease comprise myocardial infarction.

### Combined Error Minimisation

In a one-stage, or end-to-end, machine-learning algorithm, the reconstruction and classification errors are jointly optimised. This is in order to learn the best representation of z from the AE that optimises the classification error. Optimising both errors together may result in different choices of parameters for the autoencoder, for example, because the hidden layer representation which most accurately allows the decoder layers to reproduce the input may be different to the hidden layer representation that allows for the most accurate classification of the input data.

Therefore, in an embodiment where the deep neural network further comprises one or more classification layers, configured to classify the electrocardiogram data using the representation of the combined image, the deep neural network is trained by minimising a joint error calculated by combining a reconstruction error of the autoencoder layers and a classification error of the classification layers.

Previous work in the literature [29], [30] have considered a mean squared error (MSE) loss for the reconstruction error [symbol] (x, x′) when x are continuous-valued. However, it is not possible to directly combine this reconstruction loss with a classification loss. Therefore, the magnitudes of the errors may be substantially different, and one error type may dominate the optimisation. In particular, normal MSE reconstruction loss is often a number greater than 1. In such a case, optimising a direct combination of the reconstruction loss and classification loss is likely to lead to an optimisation which preferentially reduces reconstruction loss.

Therefore, in an embodiment, a normalised version of MSE for [symbol] (x, x′) is used across n data points of x. This results in a normalised reconstruction error. The normalised reconstruction error may be used in any embodiment using an autoencoder, even those using two-stage algorithms where no joint error is used, because the normalised reconstruction error can be used for optimising autoencoder performance in the same way as normal MSE error. In an embodiment, the normalised reconstruction error has a value in the range [0,1].

In an embodiment, the normalised reconstruction loss is given by

$$ℒ ( x , {x}^{′} ) = \frac{{‖ x - g ( f ( x ) ) ‖}_{2}^{2}}{{‖ x ‖}_{1} {‖ g ( f ( x ) ) ‖}_{1}} \qquad ( 4 )$$

where [symbol] (x, x′) is the normalised reconstruction error, x is a vector of the combined image comprising n datapoints, f(x) is a mapping function of the encoder layers of the autoencoder, and g(x) is a mapping function of the decoder layers of the autoencoder. ||▪||₁ is the L1 norm, and ||▪||₂ is the L2 norm. Note that [symbol] (x, x′)∈[0,1] in Equation (4). This allows for a direct comparison with other optimisation losses such as cross-entropy, which is commonly used for classification loss.

In embodiments where a joint error is used to optimise the machine-learning algorithm, the use of the normalised reconstruction error has the advantage that it can be directly combined with a classification error. In an embodiment, the reconstruction and classification losses may be added together to produce a joint error, so that the reconstruction loss in Equation (2) is changed to be a combination of reconstruction and classification losses as

[symbol]

_{T}= [symbol] _{ce}= [symbol] (x, x′) (⁹)

Alternatively, the reconstruction and classification losses may be added in quadrature. Other combinations may be chosen depending on the specific embodiment. In such embodiments, combining the reconstruction error and the classification error comprises combining the classification error with a normalised reconstruction error within the range [0, 1].

The combination of reconstruction error and classification error is not only limited to the summing of the errors directly, as shown in Equation (9). Other methods of combining the reconstruction error and classification error may also be used. For example, the errors may be added in quadrature. In some embodiments, a weighting parameter A can be introduced to ascribe different weights to the reconstruction error and the classification error. In such a case, the joint error can be calculated as

[symbol]

_{T}=λ [symbol] _{ce}+(1−λ) [symbol] (x, x′) (9a)

where λ is in the range [0, 1]. The effect of Equation (9) is the same as setting λ=0.5 in Equation (9a), which means equal weights are given to both errors (scaling each error by the same value does not change the final results).

The model is then trained to simultaneously minimise the two losses: (i) reconstruction error at the decoder and (ii) multi-class classification error. In an embodiment, the classification error may be calculated at a final softmax layer.

### DeepConvAeC

A preferred embodiment of this model is an end-to-end deep convolutional autoencoder classifier (denoted as DeepConvAEC) that leverages the characteristics of CNNs and AEs in an end-to-end deep framework that utilises both networks. DeepConvAEC incorporates the ConvAE in its feature extraction component, where convolutional layers and pooling layers are embedded in the encoder and decoder.

The architecture of this embodiment, including the feature extraction and classification components is shown in FIG. 8. It consists of two components: (i) feature extraction via convolutional autoencoder and (ii) classification via fully connected and softmax layers. The latent-space representation is constructed from these two components and is optimised simultaneously to formulate dimension-reduced features that provides the optimal accuracy in classification. Therefore, this embodiment combines the advantages of a convolutional autoencoder with the advantages of a one-stage algorithm optimised using a joint error.

As a whole, DeepConvAEC is a semi-supervised neural network trained jointly to reconstruct input data as well as optimising classification error. Its latent-space representation can be seen as a way of performing feature extraction once the weights and filters are learnt. These features can then be used to perform tasks such as classification.

DeepConvAEC uses convolutional neural networks to augment ECG data, followed by an autoencoder that learns latent features by minimising classification and reconstruction error simultaneously to extract specific features that help to improve classification. A CNN is then employed to extract features by augmenting information from ECG images. The AE jointly learns the dimensionally-reduced latent representation of the CNN features as well as the classification task simultaneously.

The end-to-end DNN enables the method to (i) to exploit abstract features describing the intrinsic relationships among ECG leads via convolutional layers; (ii) to apply unsupervised encoding of such features via AE with dimension reduction; and (iii) to target the dimension-reduced features that provides the optimal classification accuracy.

An overview of another machine-learning algorithm utilising transfer learning is shown in FIG. 9. This machine-learning algorithm is an example of a two-stage algorithm where the feature extraction and classification layers are optimised separately. In step S33, a pre-trained computer vision network (such as GoogLeNet) is used to extract hidden-layer CNN features from the input data, for example in the form of stacked spectrograms. Then, in step S35, a new hidden layer is built inside the GoogLeNet pipeline to learn ECG features. This allows the pre-existing computer vision network to be adapted to the particular ECG data used. Finally, in step S37 a classification layer, such as a softmax layer provides classification labels.

### Experimental Verification

To evaluate the performance of the method, 12 alternative embodiments were implemented. These embodiments have different combinations and choices for the machine-learning features discussed above. In the testing of these embodiments described later, the input data in all cases were processed into spectrographs and stacked according to Order-III.

The alternative embodiments are as follows.

1) Transfer Learning: To explore the potential of transfer learning, some of the embodiments used a pre-trained GoogLeNet [33] to extract CNN features from the stacked ECG spectrograms. Transfer learning approaches have the advantage of being able to take advantage of existing pre-trained neural networks, such as Inception-V3, which is used here. These pre-trained neural networks are used as the autoencoder layers, and are trained on a large quantity of generic image data from a number of sources. Classification layers are added which are trained on ECG data from patients.

CNN features were extracted particularly from the next-to-last layer of the Inception-V3 (i.e. “pool 3:0”), which provides a feature dimension of 2,048 per patient. We then performed different experiments of transfer learning on these CNN features.

I Inception-V3+SVM_{L}—extracted features from Inception-V3 and fed them into a Support Vector Machine (SVM) with a linear kernel;

II Inception-V3+SVM_{G}—same as I with a Gaussian kernel;

III Inception-V3 Classifier—Inception-V3 features were fine tuned via a dense and a softmax layers to resemble the number of classes. This is essentially the end-to-end transfer learning proposed by Xiao et al. [17];

IV Inception-V3+HL Classifier—A new hidden layer with dimension of 10 and a Rectified Linear Unit (ReLU) activation were added to Inception-V3. The features were fine tuned and classification was performed as described in III. In addition, batch normalisation was applied to the new hidden layer;

V Inception-V3+PCA Classifier—Principal Component Analysis (PCA) was applied to the CNN features derived from Inception-V3 to perform further dimension reduction. The resulting features were then fine tuned and classification was performed as described in III;

VI Inception-V3+AE Classifier—Instead of applying PCA described in V for dimension reduction, a dense AE was applied to derive the CNN features from Inception-V3. The dense AE was composed of single encoder and single decoder layers. The dimension of latent AE was optimised to 512-by-512, with a sigmoid activation on the encoder and a ReLU on the decoder;

VII Inception-V3+AE* Classifier—same as in VI but the AE is optimised for both the reconstruction loss and the classification error;

VIII Inception-V3+Convolutional AE* Classifier—same as in VII but using a convolutional AE.

2) Variants of AEs: We also experimented with different architecture of AEs as a feature extraction tool. In these embodiments, the machine-learning algorithm is trained using electrocardiogram data of a plurality of patients. Training both the autoencoder and classification layers on ECG data from patients is found to be particularly advantageous.

IX Dense AE+SVM_{G}—the same architecture of AE was used as in VI. Then dimension reduced features were fed into a SVM with a Gaussian kernel;

X Convolutional AE+SVM_{G}—the same architecture of AE was used as in VIII. Then the dimension reduced features were fed into a SVM with a Gaussian kernel;

XI Denoising convolution AE+SVM_{G}—the same as in X with an introduction of 5% additional Gaussian random noise factor in the input data.

Finally, the preferred embodiment DeepConvAeC was also implemented, for a total of 12 embodiments tested. DeepConvAeC is also trained using ECG data from patients, rather than using transfer learning.

### Data and Methods

The methods disclosed herein were validated through a study using ECG data from patients. The anonymised ECG data used in this study were collected in China. The study has obtained ethics committee approval and informed patient consent. The dataset contains 12-lead ECG waveforms from 17,381 patients (11,853 MI and 5,528 normal cases) sampled at 500 Hz. The ECG signals for each patient contain the standard 12 leads, which are I, II, III, V1, V2, V3, V4, V5, V6, aVF, aVL, and aVR.

For each ECG lead of a patient, a spectrogram was computed for a segment of 10 second window without overlap between successive windows, using the short time Fourier transform, with a Hamming window of 1 second and 95% overlaps. Each spectrogram was then re-scaled to the range of [0,1] using min-max normalisation. As the most relevant information appears in the low-frequency band of the spectrum, the first 25% of the frequency band was considered to further reduce the dimension of the spectrogram.

Different values for the dimension of the spectrograms were explored between 128 and 1024 pixels. It was found that 212-by-212 was optimal for computational, and was more convenient in reducing the feature dimension and minimising under-fitting. The detailed architecture of the specific DeepConvAEC embodiment used to obtain the results shown here can be found in Table II.

During end-to-end training of the one-stage embodiments, the Adam optimiser was used with learning rate α=0.001, training steps N=10,000, training batch size B=128. A 80% -train and 20%-test split was considered. Each experiment was repeated 10 times and mean ± standard deviation of the classification accuracy were computed. Classification accuracy is an overall performance and is defined in the usual manner as

$$\frac{T P + T N}{T P + T N + F P + F N}$$

where TP is number of MI patients that are identified as having MI, TN is the number of normal patients that are identified as normal, FP is the number of false alarms where normal patients are identified as having MI, and FN is the number of MI patients that are identified as being normal.

Other metrics of the classification are precision, sensitivity, specificity, and F-score. Precision is defined as

$$\frac{TP}{TP + FP} .$$

Sensitivity is defined as

$$\frac{TP}{TP + FN} .$$

Specificity is defined as

$$\frac{TN}{TN + FP} .$$

The F-score is defined as

$$\frac{2 TP}{2 TP + FP + FN} .$$

Due to the class-imbalance in the datasets, the sparse-softmax-cross-entropy is utilised as a classification loss. All approaches were implemented with 10-fold cross validation using the TensorFlow system [34] with Python version 3.5.

### Results

The results of the 12 embodiments, including the preferred DeepConvAEC method, are shown in Table I. With a direct use of Inception framework as a feature extraction tool for transfer learning, an accuracy of 82.8±0.0% was achieved, in a two-stage process when it was concatenated with a SVM classifier. When a dedicated neural network such as an AE was used, pairing with a SVM classifier further improved the accuracy to 86.7±0.1%.

In the case of an end-to-end approach (i.e., one stage), where the Inception features were fine tuned (i.e., Inception-V3 Classifier) it achieved a similar result as those of two stage approach using an AE. When an extra layer was added to the Inception framework (i.e., Inception-V3+HL Classifier) or further additional dimension reduction approaches (either via PCA or variants of AEs) were explored in a one stage approach, they produced lower accuracy results than vanilla Inception-V3 Classifier. This indicates that the features extracted from the Inception framework were already optimised for classification and any further dimension reduction of the extracted feature space or addition of more nodes in a hidden layer would reduce classification accuracy.

These accuracy figures represent an improvement over the approximately 70% seen in prior art methods. However, this improvement was most pronounced in the DeepConvAeC embodiment. DeepConvAEC achieved an accuracy of 94.6±0.2%, outperforming the other embodiments. DeepConvAEC had an improvement of 9.0% in accuracy when compared to the best performing of the other embodiments.

FIG. 10 shows examples of original (top row) spectrograms calculated from input data, and reconstructed spectrograms (bottom row) derived from the decoder of DeepConvAEC, where patterns of 12-lead ECGs were recovered. The four columns show examples from 1: training data using MI ECG, 2: training data from normal ECG, 3 test data from MI ECG, and 4 test data from normal ECG. In both training and testing examples, some ECG leads exhibited subtle different patterns in the MI subjects when compared to the normal subjects. Nevertheless, the method was able to learn similar details of the original spectrograms.

FIG. 11 shows a visualisation of the MI vs. Normal classes in the dense output of DeepConvAEC using a t-Distributed Stochastic Neighbour Embedding (t-SNE) algorithm. t-SNE projects high-dimensional data into a low-dimensional space of two dimensions (the x and y axes are arbitrary scales after dimension reduction by t-SNE), as shown in the figure. The data in FIG. 11 is the output of the classifier component in DeepConvAEC. This projection of the latent space into the dense output, shows that a clear classification boundary could be made in separating normal vs. MI subjects by drawing a line in the middle of the plot to separate them, thereby demonstrating the superior performance in classification when using DeepConvAEC.

As DeepConvAEC does not have 100% accuracy in separating MI cases from normal cases, the MI and normal patients are not completely spaced apart, and there are cases which might be considered as MI even though they are normal. This is to be expected from any classification algorithm, and the accuracy of DeepConvAEC is nonetheless significantly higher than other alternative methods.

TABLE I
The mean and standard deviation of accuracy of DeepConvAEC and 11 other embodiments.
The classification and feature extraction can be trained separately in two stages (i.e.,
(1) feature extraction and (2) classification) or simultaneously as a one stage end-
to-end approach (i.e., feature extraction and classification simultaneously).

| | | Accuracy | Precision | Sensitivity | Specificity | F-score |
|---|---|---|---|---|---|---|
| Design | Methods | (%) | (%) | (%) | (%) | (%) |
| Two | Inception-V₃ + SV M_{L} Classifier | 80.1 ± 0.0 | 63.2 ± 0.1 | 93.5 ± 0.1 | 73.4 ± 0.1 | 75.5 ± 0.0 |
| stages | Inception-V₃ + SV M_{G} Classifier | 82.8 ± 0.0 | 67.2 ± 0.1 | 92.5 ± 0.1 | 78.1 ± 0.1 | 77.9 ± 0.0 |
| | Two stages Dense AE + SV M_{G} | 84.4 ± 0.1 | 69.5 ± 0.2 | 93.4 ± 0.1 | 80.1 ± 0.2 | 79.7 ± 0.1 |
| | Classifier | | | | | |
| | Convolutional AE + SV M_{G} | 86.7 ± 0.1 | 73.1 ± 0.1 | 94.1 ± 0.1 | 83.2 ± 0.1 | 82.3 ± 0.0 |
| | Classifier | | | | | |
| | Denoising Convolutional | 69.2 ± 0.1 | 51.7 ± 0.1 | 90.1 ± 0.3 | 59.1 ± 0.3 | 65.7 ± 0.1 |
| | AE + SV M_{G} Classifier | | | | | |
| One | Inception-V₃ Classifier | 86.8 ± 0.1 | 77.3 ± 0.2 | 84.3 ± 0.1 | 88.0 ± 0.0 | 80.6 ± 0.0 |
| stage | Inception-V₃ + HL Classifier | 86.2 ± 0.0 | 73.2 ± 0.1 | 91.3 ± 0.2 | 83.7 ± 0.1 | 81.3 ± 0.0 |
| | Inception-V₃ + PCA Classifier | 82.9 ± 0.1 | 69.9 ± 0.0 | 84.0 ± 0.1 | 82.4 ± 0.1 | 76.3 ± 0.1 |
| | One stage Inception-V₃ + | 80.5 ± 0.1 | 63.6 ± 0.0 | 94.1 ± 0.2 | 73.8 ± 0.0 | 75.9 ± 0.0 |
| | Dense AE Classifier | | | | | |
| | Inception-V₃ + Dense AE* | 82.3 ± 0.0 | 66.9 ± 0.1 | 90.7 ± 0.1 | 78.2 ± 0.0 | 77.0 ± 0.1 |
| | Classifier | | | | | |
| | Inception-V₃ + Convolutional | 81.1 ± 0.0 | 65.4 ± 0.0 | 89.6 ± 0.2 | 76.9 ± 0.1 | 75.6 ± 0.1 |
| | AE* Classifier | | | | | |
| | DeepConvAEC* (proposed) | 94.6 ± 0.2 | 89.8 ± 0.2 | 94.6 ± 0.2 | 94.6 ± 0.2 | 92.1 ± 0.1 |

Note:
SVML and SVMG denote as SVM with linear and Gaussian kernels, respectively.

In Table I, * denotes that AE was optimised for minimising both reconstruction error and classification error (i.e. optimised to minimise a joint error combining the two different errors).

### Validation on Different Sizes of Training Set

In order to identify the minimum number of ECG cases required for providing reliable classification accuracy the number of training set cases used for training DeepConvAEC was varied from 100% to 25% of the full training data set. FIG. 12 shows the accuracy results on both the training and test sets. As mentioned above, an 80%-train and 20%-test split was considered.

It was observed that the accuracy on the same test set (i.e., 3,476 cases) had only approximately 5% performance reduction, decreasing from 94.6% to 89.5% when 13,905 (100%) and 3,476 (25%) cases were used as a training set, respectively. The results in

FIG. 12 also show that the reduction in accuracy from training to test sets are consistent across different training sizes, hence demonstrating the robustness of the model when dealing with different size of ECG cases.

### Further Comments on Results

Methods are disclosed which address issues with detecting patients with heart disease (such as myocardial infarction) in a timely manner using only electrocardiogram. The improved method of arranging input data disclosed herein achieved improvements in classification accuracy over a range of choices of different machine learning algorithms. When compared with the traditional approach of diagnosis for myocardial infarction, where both a blood test and ECG examination are required, the best performing choice of machine-learning algorithm tested herein of automated deep learning for 12-lead ECG classification of heart disease achieved an accuracy of 94.6%. Other embodiments of the machine learning algorithms also produced improved accuracy over prior art methods.

The most accurate embodiment, denoted DeepConvAEC is a deep end-to-end convolutional neural network followed by an autoencoder neural network. The framework provides an extraction of the latent dimension-reduced representation of the convolutional features that are optimised for classification. Validating on a large cohort of over 11,000

patients being diagnosed of myocardial infarction, DeepConvAEC outperformed 11 bench-marking approaches. Results show that joint minimisation of both classification and reconstruction error enhances recognition performance.

TABLE II
Architecture of the DeepConvAEC Framework. The layers in Table II
correspond to the layers in FIG. 8, except that the raw and resize
layers of the encoder, and the resize layer of the decoder are
not shown. In FIG. 8, encoder layers 1 to 10 are shown from left to
right. The latent layer (decoder layer 0) is shown centrally, connected
by solid arrows to the classification layers at the top of the figure.
Decoder layers 1 to 10 are shown from left to right following
the latent layer.

| | Layer | Type | Shape | Activation |
|---|---|---|---|---|
| Encoder | 0 | Raw | (212, 212) | |
| | 1 | 1D Convolution | (210, 210) | Sigmoid |
| | 2 | 1D Convolution | (208, 208) | Elu |
| | 3 | 1D Convolution | (206, 206) | Elu |
| | 4 | 1D Convolution | (204, 204) | Elu |
| | 5 | Maxpool | (102, 102) | |
| | 6 | 1D Convolution | (100, 100) | Elu |
| | 7 | Maxpool | (50, 50) | |
| | 8 | 1D Convolution | (48, 48) | Elu |
| | 9 | Maxpool | (24, 24) | |
| | 10 | 1D Convolution | (24, 24) | Elu |
| | 11 | Resize | (32, 32) | |
| Decoder | 0 | Latent | (32, 32) | |
| | 1 | Upsample | (64, 64) | Sigmoid |
| | 2 | 1D Convolution | (62, 62) | Elu |
| | 3 | Upsample | (128, 128) | |
| | 4 | 1D Convolution | (126, 126) | Elu |
| | 5 | Upsample | (212, 212) | |
| | 6 | 1D Convolution | (210, 210) | Elu |
| | 7 | 1D Convolution | (208, 208) | Elu |
| | 8 | 1D Convolution | (206, 206) | Elu |
| | 9 | 1D Convolution | (204, 204) | |
| | 10 | 1D Convolution | (204, 204) | Elu |
| | 11 | Resize | (212, 212) | |
| Classification | 12 | Dense | (1, 16) | Sigmoid |
| | 13 | Dense | (1, 2) | Softmax |

### REFERENCES

[1] S. Mendis, P. Puska, B. Norrving, W. H. Organization et al., Global atlas on cardiovascular disease prevention and control. Geneva: World Health Organization, 2011.

[2] S. Ansari, N. Farzaneh, M. Duda, K. Horan, H. B. Andersson, Z. D. Goldberger, B. K. Nallamothu, and K. Najarian, “A review of automated methods for detection of myocardial ischemia and infarction using electrocardiogram and electronic health records,” IEEE Reviews in Biomedical Engineering, vol. 10, pp. 264-298, 2017.

[3] J. L. Garvey, J. Zegre-Hemsey, R. Gregg, and J. R. Studnek, “Electrocardiographic diagnosis of st segment elevation myocardial infarction: an evaluation of three automated interpretation algorithms,” Journal of Electrocardiology, vol. 49, no. 5, pp. 728-732, 2016.

[4] S. Mawri, A. Michaels, J. Gibbs, S. Shah, S. Rao, A. Kugelmass, N. Lingam, M. Arida, G. Jacobsen, I. Rowlandson et al., “The comparison of physician to computer interpreted electrocardiograms on st-elevation myocardial infarction door-to-balloon times,” Critical Pathways in Cardiology, vol. 15, no. 1, pp. 22-25, 2016.

[5] P. Rajpurkar, A. Y. Hannun, M. Haghpanahi, C. Bourn, and A. Y. Ng, “Cardiologist-level arrhythmia detection with convolutional neural networks,” arXiv preprint arXiv:1707.01836, 2017.

[6] J. Zhang, S. Gajjala, P. Agrawal, G. H. Tison, L. A. Hallock, L. Beussink-Nelson,M. H. Lassen, E. Fan, M. A. Aras, C. Jordan et al., “Fully automated echocardiogram interpretation in clinical practice: feasibility and diagnostic accuracy,” Circulation, vol. 138, no. 16, pp. 1623-1635, 2018.

[7] S. W. Smith, B.Walsh, K. Grauer, K.Wang, J. Rapin, J. Li,W. Fennell, and P. Taboulet, “A deep neural network learning algorithm outperforms a conventional algorithm for emergency department electrocardiogram interpretation,” Journal of Electrocardiology, vol. 52, pp. 88-95, 2019.

[8] A. Y. Hannun, P. Rajpurkar, M. Haghpanahi, G. H. Tison, C. Bourn, M. P. Turakhia, and A. Y. Ng, “Cardiologist-level arrhythmia detection and classification in ambulatory electrocardiograms using a deep neural network,” Nature Medicine, vol. 25, no. 1, p. 65, 2019.

[9] J. Li, J. Rapin, A. Rosier, S. Smith, Y. Fleureau, and P. Taboulet, “Deep neural networks improve atrial fibrillation detection in holter. First results,” European Journal of PreventiveCardiology, vol. 23, no. 2, p. 41, 2016.

[10] S. S. Xu, M.-W. Mak, and C.-C. Cheung, “Towards end-to-end ECG classification with raw signal extraction and deep neural networks, IEEE Journal of Biomedical and Health Informatics, 2018.

[11] A. L. Goldberger, L. A. Amaral, L. Glass, J. M. Hausdorff, P. C. Ivanov, R. G. Mark, J. E. Mietus, G. B. Moody, C.-K. Peng, and H. E. Stanley, “PhysioBank, PhysioToolkit, and PhysioNet: components of a new research resource for complex physiologic signals,” Circulation, vol. 101, no. 23, pp. e215-e220, 2000.

[12] U. R. Acharya, H. Fujita, S. L. Oh, Y. Hagiwara, J. H. Tan, and M. Adam, “Application of deep convolutional neural network for automated detection of myocardial infarction using ECG signals,” Information Sciences, vol. 415, pp. 190-198, 2017.

[13] N. Strodthoff and C. Strodthoff, “Detecting and interpreting myocardial infarction using fully convolutional neural networks,” Physiological Measurement, 2018.

[14] H. W. Lui and K. L. Chow, “Multiclass classification of myocardial infarction with convolutional and recurrent neural networks for portable ECG devices,” Informatics in Medicine Unlocked, vol. 13, pp. 26-33, 2018.

[15] R. K. Tripathy, A. Bhattacharyya, and R. B. Pachori, “A novel approach for detection of myocardial infarction from ECG signals of multiple electrodes,” IEEE Sensors Journal, 2019.

[16] L. Perez and J.Wang, “The effectiveness of data augmentation in image classification using deep learning, arXiv preprint arXiv:1712.04621, 2017.

[17] R. Xiao, Y. Xu, M. M. Pelter, D. W. Mortara, and X. Hu, “A deep learning approach to examine ischemic st changes in ambulatory ECG recordings,” AMIA Summits on Translational Science Proceedings, vol. 2017, p. 256, 2018.

[18] M. M. Al Rahhal, Y. Bazi, M. Al Zuair, E. Othman, and B. BenJdira, “Convolutional neural networks for electrocardiogram classification, Journal of Medical and Biological Engineering, vol. 38, no. 6, pp. 1014-1025, 2018.

[19] D. P.Mandic and J. Chambers, Recurrent neural networks for prediction: learning algorithms, architectures and stability. John Wiley & Sons, Inc., 2001.

[20] P. Xiong, H. Wang, M. Liu, S. Zhou, Z. Hou, and X. Liu, “ECG signal enhancement based on improved denoising auto-encoder,” Engineering Applications of Artificial Intelligence vol. 52, pp. 194-202,2016.

[21] P. Xiong, H. Wang, M. Liu, F. Lin, Z. Hou, and X. Liu, “A stacked contractive denoi sing auto-encoder for ECG signal denoising,” Physiological Measurement, vol. 37, no. 12, p. 2214,2016.

[22] L. Zhou, Y. Yan, X. Qin, C. Yuan, D. Que, and L.Wang, “Deep learning-based classification of massive electrocardiography data, in 2016 IEEE Advanced Information Management, Communicates, Electronic and Automation Control Conference (IMCEC). IEEE, 2016, pp. 780-785.

[23] M. M. Al Rahhal, Y. Bazi, H. AlHichri, N. Alajlan, F. Melgani, and R. R. Yager, “Deep learning approach for active classification of electrocardiogram signals,” Information Sciences, vol. 345, pp. 340-354,2016.

[24] J. Yang, Y. Bai, F. Lin, M. Liu, Z. Hou, and X. Liu, “A novel electrocardiogram arrhythmia classification method based on stacked sparse auto-encoders and softmax regression,” International Journal of Machine Learning and Cybernetics, pp. 1-8,2017.

[25] D. Ravi, C. Wong, B. Lo, and G.-Z. Yang, “A deep learning approach to on-node sensor data analytics for mobile or wearable devices,” IEEE Journal of Biomedical and Health Informatics, vol. 21, no. 1, pp. 56-64,2017.

[26] G. Abebe and A. Cavallaro, “Inertial-vision: cross-domain knowledge transfer for wearable sensors,” in Proceedings of the IEEE International Conference on Computer Vision, 2017, pp. 1392-1400.

[27] J. Deng,W. Dong, R. Socher, L.-J. Li, K. Li, and L. Fei-Fei, “Imagenet: A large-scale hierarchical image database,” 2009.

[28] G. E. Hinton and R. R. Salakhutdinov, “Reducing the dimensionality of data with neural networks,” Science, vol. 313, no. 5786, pp. 504-507,2006.

[29] M. Ghifary, W. B. Kleijn, M. Zhang, D. Balduzzi, and W. Li, “Deep reconstruction-classification networks for unsupervised domain adaptation, in European Conference on Computer Vision. Springer, 2016, pp. 597-613.

[30] J. Liu, B. Xu, L. Shen, J. Garibaldi, and G. Qiu, “Hep-2 cell classification based on a deep autoencoding classification convolutional neural network,” in 2017 IEEE 14th International Symposium on Biomedical Imaging (ISBI 2017). IEEE, 2017, pp. 1019-1023.

[31] Y. LeCun, L. Bottou, Y. Bengio, P. Haffner et al., “Gradient-based learning applied to document recognition,” Proceedings of the IEEE, vol. 86, no. 11, pp. 2278-2324,1998.

[32] J. Masci, U. Meier, D. Cires ,an, and J. Schmidhuber, “Stacked convolutional auto-encoders for hierarchical feature extraction,” in International Conference on Artificial Neural Networks Springer, 2011, pp. 52-59.

[33] C. Szegedy,W. Liu, Y. Jia, P. Sermanet, S. Reed, D. Anguelov, D. Erhan, V. Vanhoucke, and A. Rabinovich, “Going deeper with convolutions,” in Proceedings of the IEEE Conference on Computer Vision and Pattern Recognition, 2015, pp. 1-9.

[34] M. Abadi, A. Agarwal, P. Barham, E. Brevdo, Z. Chen, C. Citro, G. S. Corrado, A. Davis, J. Dean, M. Devin, S. Ghemawat, I. Goodfellow, A. Harp, G. Irving, M. Isard, Y. Jia, R. Jozefowicz, L. Kaiser, M. Kudlur, J. Levenberg, D. Man' e, R. Monga, S. Moore, D. Murray, C. Olah, M. Schuster, J. Shlens, B. Steiner, I. Sutskever, K. Talwar, P. Tucker, V. Vanhoucke, V. Vasudevan, F. Vi' egas, O. Vinyals, P. Warden, M. Wattenberg, M. Wicke, Y. Yu, and X. Zheng, “TensorFlow: Large-scale machine learning on heterogeneous systems,” 2015, software available from tensorfloworg. [Online]. Available: https://www.tensorfloworg/

1. A computer-implemented method of classifying electrocardiogram data of a patient, comprising the steps of:
receiving input data from each of a plurality of electrocardiogram leads;
arranging the input data into a single combined image; and
applying a machine-learning algorithm to the combined image to classify the electrocardiogram data.

2. The method of claim 1, wherein the plurality of electrocardiogram leads comprises twelve leads, the twelve leads comprising three limb leads, three augmented limb leads, and six precordial leads.

3. The method of claim 2, wherein the input data are arranged in the combined image either:
in a grid of four columns and three rows, wherein:
the first column contains the input data from the three limb leads;
the second column contains the input data from the three augmented limb leads; and
the third and fourth columns each contain the input data from three of the six precordial leads; or
in a grid of four rows and three columns, wherein:
the first row contains the input data from the three limb leads;
the second row contains the input data from the three augmented limb leads; and
the third and fourth rows each contain the input data from three of the six precordial leads.

4. The method of claim 1, wherein the machine-learning algorithm comprises a deep neural network.

5. The method of claim 4, wherein the deep neural network comprises one or more autoencoder layers configured to perform feature extraction on the combined image to produce a representation of the combined image with lower dimensionality than the combined image.

6. The method of claim 5, wherein the deep neural network is trained by minimising a reconstruction error of the autoencoder layers.

7. The method of claim 5, wherein the neural network is a convolutional neural network, and the one or more autoencoder layers comprise one or more convolutional layers.

8. The method of claim 4, wherein the deep neural network comprises one or more classification layers, configured to classify the electrocardiogram data.

9. The method of claim 8, wherein the deep neural network is trained by minimising a classification error of the classification layers.

10. The method of claim 5, wherein:
the deep neural network further comprises one or more classification layers, configured to classify the electrocardiogram data using the representation of the combined image; and
the deep neural network is trained by minimising a joint error calculated by combining a reconstruction error of the autoencoder layers and a classification error of the classification layers.

11. The method of claim 10, wherein combining the reconstruction error and the classification error comprises combining the classification error with a normalised reconstruction error within the range [0, 1].

12. The method of claim 11, wherein the normalised reconstruction error is given by:
$$ℒ ( x , {x}^{′} ) = \frac{{‖ x - g ( f ( x ) ) ‖}_{2}^{2}}{{‖ x ‖}_{1} {‖ g ( f ( x ) ) ‖}_{1}}$$
where: [symbol]
(x, x′) is the normalised reconstruction error;
x is a vector of the combined image comprising n datapoints;
f(x) is a mapping function of the encoder layers of the autoencoder; and
g(x) is a mapping function of the decoder layers of the autoencoder.

13. The method of claim 1, wherein the machine-learning algorithm is trained using electrocardiogram data of a plurality of patients.

14. The method of claim 1, wherein the machine-learning algorithm is configured to classify the electrocardiogram data into one of two or more categories, the two or more categories comprising normal heart activity and one or more categories of disease.

15. The method of claim 14, wherein the one or more categories of disease comprise myocardial infarction.

16. The method of claim 1, wherein the step of arranging the input data into a single combined image comprises:
processing the input data to produce a spectrogram of the spectrum of frequencies of the ECG signal derived from each of the plurality of electrocardiogram leads; and
arranging the spectrograms into a single combined image.

17. The method of 1, wherein the step of arranging the input data into a single combined image further comprises normalising the input data.

18. An apparatus for classifying electrocardiogram data of a patient comprising:
receiving means configured to receive input data from each of a plurality of electrocardiogram leads;
processing means configured to arrange the input data into a single combined image; and
classification means configured to apply a machine-learning algorithm to the combined image to classify the electrocardiogram data.

19. A computer program comprising instructions which, when the program is executed by a computer, cause the computer to carry out the method of claim 1.

20. A computer-readable medium comprising instructions which, when executed by a computer, cause the computer to carry out the method of claim 1.
